(19) United States
(12) Patent Application Publication
(10) Pub. No.: US 2010/0158983 A1
(43) Pub. Date: Jun. 24, 2010

(54) METHOD FOR INCREASING THE PERMEABILITY OF POLYMER FILM

(76) Inventors: Thomas A. Davis, El Paso, TX (US); Walker M. Rast, Colubia, SC (US)

Correspondence Address: DORITY & MANNING, P.A., POST OFFICE BOX 1449, GREENVILLE, SC, 29602-1449, US

(21) Appl. No.: 12/162,667

(22) PCT Filed: Feb. 7, 2007
(86) PCT No.: PCT/US07/03138

§ 371 Date: Feb. 23, 2010

Related U.S. Application Data
(60) Provisional application No. 60/765,918, filed on Feb. 7, 2006.

(51) Int. Cl. C08J 5/20; A61F 2/16; H01M 8/10; C25B 13/00; B32B 5/22; A61F 13/02

(52) U.S. Cl. 424/429; 521/27; 429/492; 204/296; 442/76; 602/43

(57) ABSTRACT

Polymer membranes are disclosed having increased permeability. The process of the present disclosure, for instance, can increase the ion permeability of membranes and/or the gas permeability of membranes. In one embodiment, for instance, a precursor polymer is subjected to energy in an amount sufficient to form damage tracks through the thickness of the polymer. The damage tracks are then oxidized to form free radical groups. The precursor polymer is then hydrolyzed causing ion groups to form that cluster along the damage tracks. In one embodiment, sulfonated tetrafluoroethylene-based copolymer ionomer membranes are formed that have increased conductivity. Other ionomer membranes that may be formed according to the present disclosure include copolymers of a vinyl hydrocarbon and a vinyl carboxylic acid.

### CROSS-REFERENCE TO RELATED APPLICATIONS

The present application is based upon and claims priority to U.S. Provisional Patent Application having Ser. No. 60/765,918, filed on Feb. 7, 2006.

### BACKGROUND

(1) Field of the Invention

The present invention relates to a new method for increasing the permeability of polymer films.

(2) Description of the Related Art

Nafion® membranes marketed by the Dupont de Nemours Company and similar perfluorinated, ion-permeable membranes are important components in many processes that employ electrochemistry. They are used as separators in chlor-alkali cells and as proton transport media in fuel cells. In these and other applications it is beneficial to have the ionic conductivity through the membrane as high as possible. Anisotropic conductance of Nafion® 117 membranes is about 8.56×10⁻² S/cm in the plane of the membrane and 2.4×10⁻² S/cm normal to the plane of the membrane. The measured anisotropy is opposite of what is desired in many applications. For instance, if ion-permeable membranes could be made more conductive in the direction normal to the membrane, the power output of a PEM fuel cell would be increased and the energy efficiency of an electrochemical cell would be improved.

The present disclosure introduces novel methods of increasing the ionic conductivity of the membranes to overcome the disadvantages of prior art constructions and methods. In addition, the present disclosure is also directed to similarly treating any suitable ionomer membranes. For instance, in other embodiments, the process of the present disclosure can be used to increase the gas permeability of the membranes. Polymer membranes made according to the present disclosure may be used in numerous applications, in addition to being used as an ion exchange membrane.

### SUMMARY

Perfluorinated ion-permeable membranes, such as Nafion® membranes, are commonly used as proton transfer media in fuel cells, as cation permeable separators in electrochemical cells and as coatings for sensors. In these and most other applications, it is beneficial to have the ionic conductivity through the membrane as high as possible. However, it has been reported that ion-permeable membranes are substantially more conductive in the plane of the membrane than in the direction normal to the plane. The present disclosure introduces a method for increasing conductivity in the direction perpendicular to the plane of the membrane. This technology for controlling ion permeability can be applied to the control of gas permeability of certain polymers, particularly ion-containing polymers, and can potentially be used to make gas-permeable products, such as contact lenses or wound dressings.

For instance, in one embodiment, the present disclosure is directed to a membrane comprising an ionomer. The ionomer contains ionic groups and non-polar polymer chains. In accordance with the present disclosure, the membrane defines damage tracks comprising broken polymer chains in association with peroxy free radical groups. The damage tracks extend through at least a portion of the thickness of the membrane. By forming the damage tracks, the ionic groups become clustered along the damage tracks thereby increasing the conductivity of the membrane in a direction normal to a major surface of the membrane and/or thereby increasing the gas and/or vapor permeability of the membrane. For instance, the gas permeability of the membrane can be increased while still remaining liquid impermeable.

In general, any suitable ionomer may be used to make the membrane. The ionomer may comprise, for instance, a perfluorinated polymer containing sulfonyl fluoride groups, such as a sulfonated tetrafluoroethylene-based copolymer. In an alternative embodiment, the ionomer may comprise a copolymer of a vinyl hydrocarbon and a vinyl carboxylic acid containing carboxylate groups. The copolymer may comprise, for instance, a poly(ethylene-co-methacrylic acid). The ionomer may contain any suitable ionic groups, such as sulfonate groups or carboxylate groups.

The membrane can take different physical forms depending upon the particular application. For instance, the membrane can comprise a film or a coating. The membrane can be used as an ion exchange membrane, such as a proton exchange membrane. Alternatively, the membrane can be used to form various articles where gas permeability is desired. For instance, the membranes can be used to form contact lenses or can be used to form coatings on articles of clothing, such as coats and the like. The ionomer coatings, for instance, can be produced so as to be gas permeable, while remaining liquid impermeable.

In order to form the ionomer membranes, in one embodiment, an ionomer precursor polymer membrane can be subjected to sufficient amounts of energy to form damage tracks where polymer chains have been broken. The damage tracks extend through at least a portion of the thickness of the membrane.

After or during the formation of the damage tracks, the broken polymer chains are oxidized. The polymer chains are oxidized by exposing the damage tracks to an atmosphere containing oxygen. Oxidation of the broken polymer chains can form, for instance, peroxide free radical groups.

The ionomer precursor polymer membrane containing the damage tracks is then hydrolyzed to form an ionomer containing ionic groups and non-polar polymer chains. The ionic groups become clustered along the damage tracks. In this manner, the ionic conductivity and/or gas permeability of the membrane through its thickness can be increased.

The energy used to form the damage tracks can vary depending upon the particular application. For instance, in one embodiment, the membrane can be subjected to ionizing radiation. In an alternative embodiment, the membrane can be subjected to electrical discharges. The radiation and/or the electrical discharges are present in an amount sufficient to form the damage tracks without severely degrading the physical properties of the membrane.

As described above, the damage tracks penetrate through at least a portion of the thickness of the membrane. For instance, in one embodiment, the damage tracks can penetrate through at least about 40% of the thickness of the membrane, such as at least about 60% of the thickness of the membrane, such as at least about 80% of the thickness of the membrane. For example, in one embodiment, at least some of the damage tracks penetrate all the way through the thickness of the membrane. Once the ionomer precursor polymer membrane is hydrolyzed, the damage tracks form ionic channels within the membrane.

The ionomer precursor polymer membrane can be hydrolyzed using any suitable hydrolyzing agent. For instance, in one embodiment, a strong base may be used to hydrolyze the polymer. The strong base, for instance, may comprise a hydroxide, such as sodium hydroxide. Hydrolyzing the polymer forms the ionic groups. For instance, in one particular embodiment, sulfonyl groups are converted into sulfonate groups.

In certain embodiments, the resulting ionomer membrane can be further contacted with other compositions in order to incorporate a chemical additive into the membrane. For instance, in one embodiment, the ionomer membrane can be contacted with a composition containing a functional chemical. The functional chemical may become located along the damage tracks in association with the ionic groups. The functional chemical may comprise, for instance, a dye or an optical agent that changes the optical properties of the membrane.

Other features and aspects of the present disclosure are discussed in greater detail below.

### BRIEF DESCRIPTION OF THE DRAWINGS

A full and enabling disclosure of the present invention, including the best mode thereof to one skilled in the art, is set forth more particularly in the remainder of the specification, including reference to the accompanying figures, in which:

FIG. 1 is a diagram of one embodiment of a process for synthesizing sulfonated tetrafluoroethylene-based copolymer and is provided for purposes of explanation; and

FIG. 2 is a demonstrative diagram illustrating a sulfonated tetrafluoroethylene-based copolymer in an aqueous medium.

### DETAILED DESCRIPTION

It is to be understood by one of ordinary skill in the art that the present discussion is a description of exemplary embodiments only, and is not intended as limiting the broader aspects of the present invention.

In general, the present disclosure is directed to ionomer membranes and to processes for forming the membranes. As used herein, the term “membrane” includes polymeric objects with a thickness that is small compared to length and width and includes films, coatings, lenses, and other objects. In accordance with the present disclosure, ionomer membranes can be formed having improved physical and/or chemical properties. For instance, ionomer membranes can be made according to the present disclosure having improved conductivity through the thickness of the membrane. Ionomer membranes can also be formed having increased gas permeability characteristics.

Of particular advantage, the conductivity and/or the permeability of a membrane can be increased in a very controlled manner. For example, the conductivity of a membrane can be increased according to the present disclosure without substantially changing the gas permeability characteristics of the membrane. Further, when it is desired to increase the gas permeability characteristics of the membrane, the permeability can be increased in controlled amounts.

In order to produce membranes in accordance with the present disclosure, a film comprising a precursor polymer is subjected to sufficient amounts of energy to cause damage tracks to form at least partially through the thickness of the film. The energy contacted with the film may comprise, for instance, any suitable form of radiation or, alternatively, electric discharges. In one embodiment, the energy may be at a particular level or may be focused so that damage tracks are formed in the film without substantially degrading the properties of the film.

The damage tracks that are formed primarily comprise areas in the film where polymer chains have been broken, and the exposed ends of the broken polymer chains contain radical groups. The radical groups that form may depend upon the precursor polymer. In one embodiment, for instance, peroxide radical groups may form when the ends of the broken polymer chains are exposed to oxygen.

In order to convert the precursor polymer into an ionomer, the precursor polymer is hydrolyzed. Hydrolyzing the precursor polymer forms ionic groups. For instance, the ionic groups may comprise sulfonate groups or carboxylate groups. The ionic groups are contained in the ionomer in conjunction with non-polar polymer chains. The ionic groups tend to cluster within the polymer. According to the present disclosure, the ionic groups have a tendency to cluster within the damage tracks due to the presence of the free radical groups. The damage tracks thus form areas where the permeability of the film is increased. For instance, the damage tracks form ionic channels for increasing the conductivity of the film through its thickness. In addition, the damage tracks can also increase the gas permeability characteristics of the film.

In general, any suitable ionomer may be made according to the present disclosure. The ionomer may comprise, for instance, an ethylene-based ionomer, a styrene-based ionomer, a fluoropolymer-based ionomer, a carboxylated-nitrile-based ionomer, a rubber-based ionomer and the like.

In one embodiment, for instance, the ionomer may comprise a copolymer of a vinyl hydrocarbon and a vinyl carboxylic acid containing carboxylate groups. In this embodiment, for instance, the ionomer may comprise a poly(ethylene-co-methacrylic acid) copolymer or a polyethylene-co-acrylic acid) copolymer.

In still another embodiment, the ionomer may be formed from an ethylene/styrene sulfonic acid metal salt, such as a sodium salt.

In still another embodiment, the ionomer may be formed from a perfluorinated polymer containing sulfonyl fluoride groups and/or carboxyl groups. For instance, in one embodiment, the ionomer may comprise a polymer with a poly(tetrafluoroethylene) (PTFE) backbone and with regular side chains ending in ion exchange groups SO₃⁻ or COO⁻. The above ionomers, for instance, are currently sold under the name Nafion® and are available from Dupont. Nafion® membranes, for instance, can be used in electrochemical cells.

For purposes of explanation, the following is a description of a process for producing ionomer membranes in accordance with the present disclosure made from a perfluorinated polymer, and particularly from a sulfonated tetrafluoroethylene-based copolymer that is used to form Nafion® membranes. Since the COO⁻ group is normally present only on a surface of Nafion® membranes used in electrochemical cells, descriptions that follow will refer to Nafion® with SO₃⁻ groups, but also apply to Nafion® with any ion exchange group. In fact, the following process is also applicable to any suitable ionomer.

Sulfonated tetrafluoroethylene-based copolymers are made by copolymerization of CF₂═CF₂ and one or more monomers represented by the structure CF₂═CFY where Y is a chain of perfluorinated alkanes with ether linkages interspersed and terminated with sulfonyl fluoride —SO₂F. The sulfonyl fluoride form of the polymer, called XR resin, is a precursor polymer that is melt extruded into membranes, which are subsequently treated with NaOH solution to hydrolyze the —SO₂F to —SO₃⁻. Another method of forming sulfonated tetrafluoroethylene-based copolymer films is to dissolve the —SO₃⁻-form polymer in a solvent and cast a film. This method is usually used for coating objects, such as electrodes, with a thin layer of the polymer.

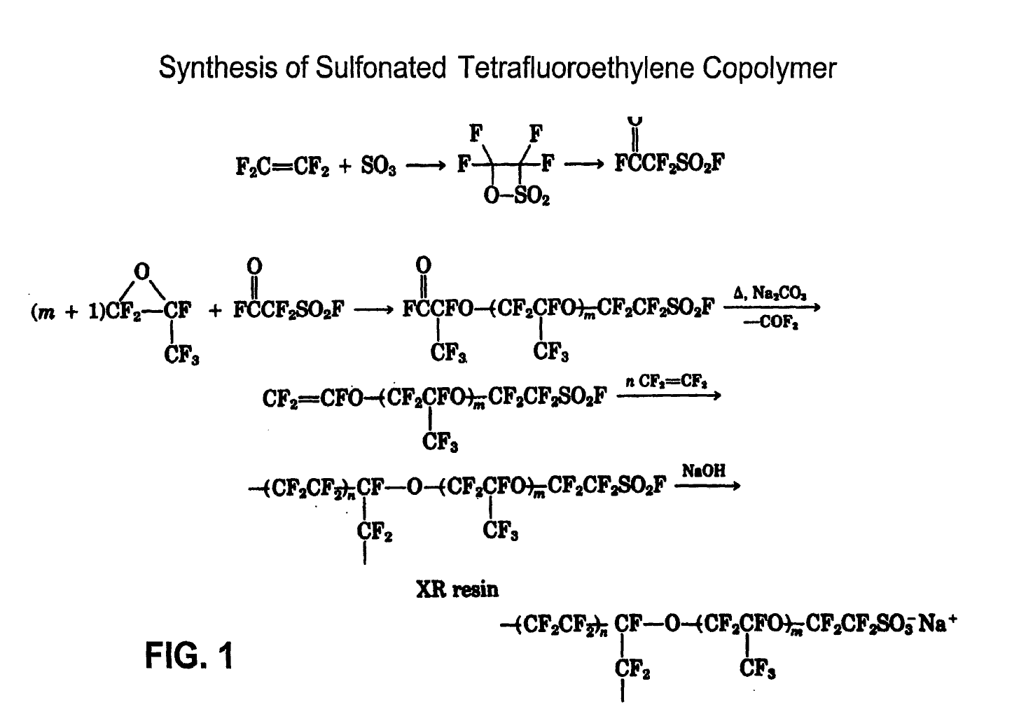

Referring to FIG. 1, synthesis of a sulfonated tetrafluoroethylene-based copolymer is shown. As shown in this embodiment, a fluoropolymer containing sulfonyl groups is copolymerized with a monomer having the structure CF₂═CF₂ to form an ionomer precursor polymer also known as the XR resin. The XR resin is then hydrolyzed with a base, such as sodium hydroxide, to form the ionomer. The ionomer contains ion groups comprised of sulfonate groups.

After the membranes are in the —SO₃⁻ form they can no longer be melt processed. It has been observed in small angle X-ray scattering studies that the XR resin has crystal structure very similar to that of PTFE. This fact implies a substantial change in the nanostructure as the —SO₂F is converted to —SO₃⁻.

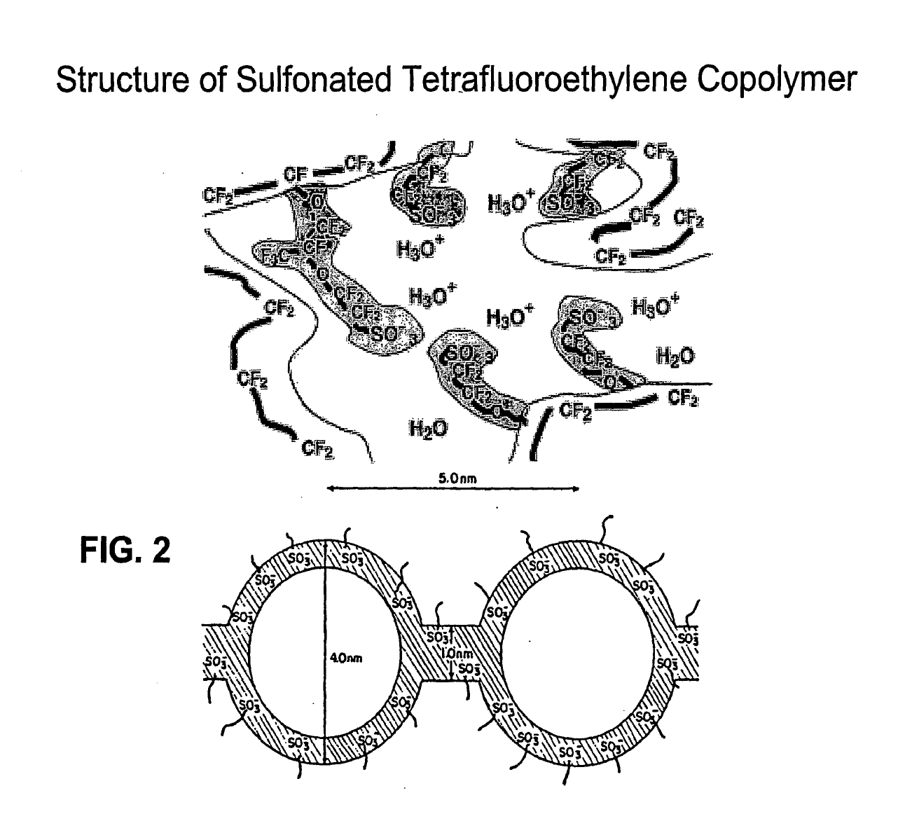

Sulfonated tetrafluoroethylene-based copolymer is an ionomer, a class of polymer in which the ionic groups tend to cluster because of incompatibility between the polar ionic groups and the non-polar polymer chains. The special feature about Nafion® that makes it a good ion-permeable membrane is that the clusters are linked by channels that allow ions to move from one cluster to another as they migrate through the membrane in response to an electrical potential across the membrane. The charge density of ions in these clusters is so high that Nafion® should be even more conductive than it actually is. The reason for its limited conductivity is that the channels connecting the clusters are randomly oriented. For instance, referring to FIG. 2, an illustration is included showing the structure of a sulfonated tetrafluoroethylene-based copolymer. As shown in the top of the figure, the ionic sulfonate groups form clusters among the non-polar polymer chains. These clusters tend to be randomly dispersed throughout the thickness of the polymer. As shown in the bottom of FIG. 2, the clusters tend to be connected by ionic channels. For example, in one characterization, the ionic clusters may have a diameter of approximately 4 nanometers, and the connecting channels have a diameter of approximately 1 nanometer. Multiple channels may form in parallel and may be connected or otherwise associated with each other so as to form a “dumbbell-like” shape. The ionic channels that form in the ionomer may extend in any direction. The present disclosure is directed to orienting the ionic channels in a direction substantially perpendicular to the surface of the polymer so that the ionic channels extend through a substantial portion of the thickness of the polymer. By orienting the ionic channels through the thickness of the polymer, the resulting polymer can become more conductive to the migration of ions.

In accordance with the present disclosure, the ionic channels are oriented within an ionomer membrane, such as a sulfonated tetrafluoroethylene-based copolymer membrane, by forming damage tracks through the thickness of a precursor polymer. Damage tracks are formed through the thickness of the polymer by subjecting the polymer to energy in controlled amounts. When subjected to energy in accordance with the present disclosure, polymer chains become broken forming the damage tracks. The ends of the broken polymer chains are then oxidized by exposing the polymer to oxygen and thereby causing the formation of peroxy free radical groups. The polar free radical groups along the damage track then become preferred sites for formation of channels lined with ion groups, such as the sulfonate groups, during conversion of the precursor polymer into the ionomer. The precursor polymer is converted into an ionomer, for instance, by hydrolyzing the polymer to form the ionic groups.

A “track-etch” method was first used to make holes in flakes of mica. Later, microfiltration membranes were produced by Nuclepore Corp. by radiation with thermal neutrons to form “damage tracks” in a polycarbonate film. These tracks were then etched out with NaOH solution to form cylindrical pores through the thickness of the Nuclepore membrane. The present inventors, however, are not aware of a process in which damage tracks have been formed in a precursor polymer prior to formation of an ionomer.

The manner in which the damage tracks are formed in the precursor polymer depends upon various factors including the type of precursor polymer being treated and the desired results. In general, any suitable energy source capable of forming damage tracks in a controlled manner may be used. In one embodiment, for instance, the damage tracks may be formed by electrical discharges. The electrical discharges, for instance, can be focused at localized areas for forming damage tracks at particular locations. It is believed that electrical discharges can be scanned across a polymer membrane made from a precursor polymer in order to form damage tracks that have a desired length and appear in the final product according to a desired density (damage tracks per unit area).

In an alternative embodiment, the damage tracks may be formed by subjecting the precursor polymer to radiation in controlled amounts. The term “radiation” is used herein to mean the exposure of the membrane to any type of particles, atomic or subatomic, or energy beam of sufficient energy to pass substantially through at least a portion of the thickness of the membrane. The radiation may comprise, for instance, ionizing radiation in which heavy ions are caused to strike a surface of the precursor polymer. The heavy ions, for instance, may be produced using an accelerator. An accelerator produces a beam of high-speed heavy ions by passing an electron beam through a sputter electrode containing a magnetic field. The electron beam passes an anode and forms a beam that then passes through an extraction electrode and through a mass separator to form a beam of heavy ions. The heavy ions can then be scanned across the surface of a precursor polymer at levels of energy sufficient to form damage tracks, but at levels insufficient to substantially degrade the mechanical properties of the precursor polymer. In one embodiment, for instance, the level of energy of the heavy ions can be less than about 20 meV, such as less than about 15 meV, such as from about 2 meV to about 15 meV. For example, the energy levels may be from about 8 meV to about 12 meV, such as about 10 meV.

As described above, the damage tracks are formed through the thickness of the polymer in a direction that can be substantially perpendicular to a major surface of the polymer membrane. The damage tracks, however, can form at any suitable angle to the surface of the membrane as long as the damage tracks substantially penetrate through the thickness of the polymer. In general, the damage tracks can extend through at least about 40% of the thickness of the membrane, such as at least about 50% of the thickness of the membrane, such as at least about 60% of the thickness of the membrane, such as at least about 70% of the thickness of the membrane, such as at least about 80% of the thickness of the membrane, such as at least about 90% of the thickness of the membrane. In some applications, it may be most desired that at least certain of the damage tracks extend all the way through the thickness of the membrane.

The amount the damage tracks penetrate through the thickness of the polymer can be varied depending upon the particular application. For instance, in some applications, it may be desirable to increase the conductivity of the membrane without substantially influencing the gas permeability characteristics of the membrane. In this embodiment, it may be desirable not to have the damage tracks extend entirely through the membrane. For instance, in this embodiment, the damage tracks may extend from about 40% to about 80% through the thickness of the membrane. In one embodiment, the membrane can be subjected to energy on opposing sides so that the damage tracks are initiated from both sides of the membrane. The opposing damage tracks may provide ionic channels for increasing the conductivity of the membrane without substantially interfering with the gas permeability characteristics of the membrane.

In other embodiments, however, it may be desirable to increase the gas permeability characteristics of the membrane. In this embodiment, the damage tracks may extend all the way through the thickness of the membrane. The amount of damage tracks that are formed in the membrane may be used to control the amount of increase in the gas permeability characteristics.

As described above, in one embodiment, ionizing radiation may be used to form the damage tracks. It should be understood, however, that any suitable form of radiation may be used.

Damage tracks formed through the precursor polymer in the —SO₂F form are believed to become preferred paths for the formation of ionic channels in the ionomer polymer. It is known that clusters form during annealing of solution-cast sulfonated tetrafluoroethylene-based copolymer films, so damage tracks in solution-cast membranes should also influence the orientation of the channels. If damage tracks are produced while the ionic clusters and channels are being formed in the manufacture of ionomer membranes, then the channels would tend to form along these tracks instead of forming entirely randomly in all directions.

Perfluorinated polymers (PTFE in particular) are known to be susceptible to radiation damage. X-rays directed onto Nafion® membranes in H and Na ion forms have shown that polymer chains are broken. Electron paramagnetic resonance (EPR) spectroscopy analysis of the irradiated membranes that were subsequently exposed to air revealed peroxy free radicals —CF₂CF(OO.)CF₂— and —CF₂CFOO.. Further their analysis with photoacoustic FTIR spectroscopy and with Raman scattering indicated the formation of C═O polar groups in the irradiated Nafion® membrane.

The reported studies on radiation effects on Nafion® membranes seem to be “postmortem” determinations of the damage caused by radiation, and they were often done at high levels of radiation that caused discoloration and deterioration of the mechanical properties of the samples. The present inventors, however, are not aware of any previously reported process in which an ionomer precursor polymer was subjected to lower doses of radiation in order to form damage tracks for increasing the proton-transport properties of the resulting ionomer membrane. It is believed that radiation at dosages well below severely detrimental level will form damage tracks that lead to the formation of hydrophilic peroxy free radicals in the —SO₂F-form films that will become the preferred sites for the formation of the hydrophilic —SO₃⁻Na⁺ clusters and channels in the final ionomer membrane. When the path of the radiation is perpendicular to the surface of the film, the trans-membrane proton conductivity of the resulting membrane should be substantially improved.

Others have described the use of radiation for the purpose of grafting ionic groups to polymer films or for the purpose of cross linking the polymer, but the concept of radiation of a precursor polymer for the purpose of aligning the ion-conducting channels has not been disclosed heretofore. U.S. Pat. Nos. 6,902,849 and 5,356,386 describe irradiation of a polymer in order to link sulfonic acid groups thereto. U.S. Pat. Nos. 5,830,921, 4,230,549 and 4,339,473 describe irradiation of perfluorinated polymers for the purpose of grafting vinyl monomers, which can be subsequently sulfonated. U.S. Pat. Nos. 5,994,426 and 5,643,968 describe irradiation to impart cross linking. U.S. Pat. No. 5,128,014 describes irradiation with an electron beam accelerator to cause a reduction in electrical resistance during electrolysis, but those membranes were already converted to the ionic form before the irradiation.

Membranes of the —SO₂F precursor form can be submitted to a variety of forms of radiation in accordance with the present disclosure and then hydrolyzed to form the ionomer. After being processed according to the present disclosure, the ionomer has increased electrical conductivity in comparison to membranes that were formed without being subjected to radiation. The gas permeability of membranes made according to the present disclosure may also be increased. Further, these properties can be changed without substantially interfering with the mechanical strength of the polymer.

The precursor polymer that is subjected to energy in accordance with the present disclosure can be formed in various ways. For instance, the precursor polymer can be extruded into a membrane or may be cast from a solution containing the precursor polymer.

Membranes first cast from a precursor polymer solution do not have a fully-developed network of clusters and channels. This network is developed by annealing the cast membrane. When radiation is passed through the membrane before or during the annealing operation, channels form preferentially along the damaged tracks and result in a membrane with higher conductivity than a membrane annealed without radiation. Indeed, radiation of the fluorinated polymer during the manufacture of the membrane results in higher conductivity of that membrane.

The present methods can also be demonstrated by radiating membranes that have been solution-cast onto metal surfaces and onto release surfaces. The membranes are then annealed by standard methods. The released membranes can have increased conductivity and/or permeability through the thickness of the membrane.

As described above, irradiation during the hydrolysis of extruded films facilitates formation of channels normal to the plane of the membrane. Formation of channels normal to the plane of the membrane can also be facilitated by application of an electric potential during the hydrolysis of extruded films.

Fluoropolymer ionomer membranes, such as sulfonated tetrafluoroethylene-based copolymer membranes, made in accordance with the present disclosure have numerous applications based upon the increased conductivity over the thickness of the polymer. For instance, membranes made in accordance with the present disclosure allow reduced power consumption during electrolysis. The membranes may increase power output when used in fuel cells.

It should be understood that the process of the present disclosure can be used to modify any suitable ionomer membrane, in addition to fluoropolymer ionomers. In fact, ionomers are used in numerous applications, in addition to being used as ion permeable membranes. For instance, ionomers can have high impact strength at low temperature and can be puncture and abrasion resistant. The precursor polymers used to form ionomers can have high melt elasticity and good thermal forming properties. In addition, the polymers can have a relatively low sealing temperature and can have a relatively high sealing seam strength. Ionomers are also known to be resistant to grease, oil and solvents.

The process of the present disclosure can be used to modify different properties of ionomer polymers in addition to increasing conductivity. For instance, orienting the channels between the ion clusters in ionomers allows the membranes to be more permeable to gases as well as ions. Increasing the gas permeable properties of membranes may be useful in numerous applications. For instance, ionomers made in accordance with the present disclosure having improved gas permeability characteristics may be useful to construct contact lenses, wound dressings, protective clothing including footwear, glass coatings, abrasion-resistant coatings. The channels formed in ionomers can also be used as templates for nano-scale devices. When forming the above products, the ionomer polymers made in accordance with the present disclosure may be used to construct the device itself or may be comprised of a coating on the article. For instance, ionomers made in accordance with the present disclosure may be a coating on a wound dressing or a coating on an article of clothing. The coating can be water impermeable while remaining permeable to gas and vapor.

It is believed that ionomers made according to the present disclosure are particularly well suited for producing contact lenses. Through the process of the present invention, the ionomer polymers will have increased gas permeability for use in contact lens applications. Further, since the ion channels formed in membranes made according to the present invention extend in a direction perpendicular to the surface of the polymer, it is believed that the ion channels do not interfere with the optical clarity of the material. Ion channels that extend in a direction parallel to the plane of the membrane, on the other hand, may adversely interfere with the optical clarity of the polymer.

In some applications, it may also be beneficial to incorporate into the ionomer an additional composition that collects in the ion channels. For instance, in one embodiment, a composition containing a chemical agent, such as a dye, may be contacted with an ionomer made in accordance with the present disclosure causing the dye to concentrate within the ion channels. For instance, a dye might be used that may be attracted to the ions that extend along the channels. When used in a contact lens application, the perpendicular channels loaded with dyes may add color to the contact lens that would be visible to everyone except the wearer. In other embodiments, the ion channels may be loaded with materials that absorb light that enters the lens from an angle for wearers that want to reduce peripheral distractions or glare. Channels may also serve as reservoirs of therapeutic agents to prevent irritations, infections and deterioration of the eye. As described above, the channels may also serve as templates for preparation of nano-scale devices.

Other ionomer polymers that may be formed according to the present disclosure include ethylene-based ionomers, styrene-based ionomers, carboxylated nitrile-based ionomers, rubber-based ionomers, and the like. For instance, in one embodiment, an ethylene/styrene sulfonic acid sodium salt ionomer may be formed according to the present disclosure.

In an alternative embodiment, the ionomer may be formed from a copolymer of a vinyl hydrocarbon and a vinyl carboxylic acid. For instance, in one embodiment, the ionomer may comprise an acrylic acid copolymer or a methacrylic acid copolymer.

In one particular embodiment, for instance, the ionomer may comprise a poly(ethylene-co-methacylic acid) copolymer having the following formula:

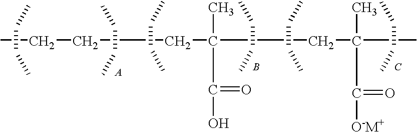

wherein A is the portion of ethylene in the monomer mix, the sum of B and C is the portion of methacrylic acid in the monomer mix, and C is the portion for which the cation M has been exchanged for H. In one embodiment, for instance, A, B, and C are such that the copolymer contains from about 85% to about 95% by weight ethylene and from about 15% to about 5% by weight methacrylic acid. For instance, in one particular embodiment, the copolymer may contain about 91% by weight ethylene and about 9% by weight methacrylic acid, and wherein M is a cation, such as hydrogen or a metal. For example, M can be sodium, potassium, lithium, zinc, magnesium and the like.

Ionomers made according to the above chemical formula are sold under the tradename Surlyn® by the Dupont de Nemours Company. As shown above, the poly(ethylene-co-methacrylic acid) copolymer ionomer contains carboxylate groups as the ion groups.

In another embodiment of the present disclosure, the ionomer comprises a poly(ethylene-co-acrylic acid) copolymer ionomer. Such polymers are sold under the tradename ESCOR and typically also contain carboxylate groups. The carboxylate groups can be neutralized with any suitable cation, such as those listed above. For instance, in one embodiment, the polymer is zinc-neutralized.

In still another embodiment of the present disclosure, the ionomer comprises a poly(ester-sulfonic acid) copolymer. Such copolymers are commercially available from the Eastman Chemical Company under the tradename AQxxy, wherein the xx are digits and the y is a letter.

In still another embodiment, the ionomer made according to the present disclosure is a zinc-neutralized sulfonated poly(2,6-dimethyl-1,4-phenylene oxide) ionomer. Such ionomers are commercially available from General Electric.

Polymer ionomer membranes made according to the present disclosure can have any suitable thickness depending upon the particular application. For instance, the membranes can have a thickness of less than about 5 mm, such as less than about 4 mm, such as less than about 3 mm, such as less than about 2 mm, or even less than 1 mm. For instance, the membranes can have a thickness of from about 0.001 mm to about 3 mm.

The scope of this disclosure is also not intended to be limited to the modification of polymer films that already have ionic or ionogenic groups attached to the polymer chain. Falling within the scope this invention is the radiation, in a direction substantially perpendicular to the plane of the film, and subsequent treatment of the irradiated film to form functional groups that are substantially oriented along the path of the damage tracks that cause the local properties of the material along that path to be different from the properties of the bulk polymer. For example, if the procedure for forming the track-etch Nuclepore microfiltration membrane had included a step of treating the damage tracks with a reagent that formed ion-exchange groups along the path, then that modified procedure would fall within this invention. It should be noted that this invention does not require that the damage track or the path of modified properties extend through the entire thickness of the film.

It is to be understood that the foregoing description of the invention and other modifications and variations to the present invention may be practiced by those of ordinary skill in the art, without departing from the spirit and scope of the present invention. In addition, it should be understood that aspects of the various embodiments may be interchanged both in whole or in part. Furthermore, those of ordinary skill in the art will appreciate that the foregoing description is by way of example only, and is not intended to limit the invention.

What is claimed:

1. A process for forming ionomer membranes comprising:
subjecting an ionomer precursor polymer membrane with sufficient amounts of energy to form damage tracks where polymer chains have been broken, the ionomer precursor polymer membrane having a thickness, the damage tracks extending through at least a portion of the thickness of the membrane; and
hydrolyzing the ionomer precursor polymer membrane containing the damage tracks to form an ionomer containing ionic groups and non-polar polymer chains, the ionic groups being clustered along the damage tracks.

2. A process as defined in claim 1, wherein the ionic groups comprise sulfonate groups or carboxylate groups.

3. A process as defined in claim 1, wherein the energy that the membrane is subjected to comprises ionizing radiation.

4. A process as defined in claim 1, wherein the energy that the membrane is subjected to comprises electrical discharges.

5. A process as defined in claim 1, wherein at least a portion of the damage tracks extend through at least about 40% of the thickness of the membrane.

6. A process as defined in claim 1, wherein at least some of the damage tracks extend all the way through the thickness of the membrane, the ionic groups forming ionic channels along the damage tracks.

7. A process as defined in claim 1, wherein the ionomer precursor polymer membrane comprises a perfluorinated polymer containing sulfonyl fluoride groups.

8. A process as defined in claim 7, wherein the ionomer has increased conductivity through the thickness of the membrane in comparison to a similarly formed ionomer not subjected to the energy.

9. A process as defined in claim 1, wherein the ionomer precursor polymer membrane comprises a copolymer of a vinyl hydrocarbon and a vinyl carboxylic acid.

10. A process as defined in claim 1, wherein the membrane comprises a film or a coating.

11. A process as defined in claim 1, wherein the ionomer precursor polymer membrane is hydrolyzed by being contacted with a strong base.

12. A process as defined in claim 11, wherein the strong base comprises a hydroxide.

13. A process as defined in claim 1, wherein the ionomer is subsequently contacted with a composition containing a functional chemical, the functional chemical being localized along the damage tracks in association with the ionic groups.

14. A process as defined in claim 1, wherein the precursor polymer membrane comprises poly(ethylene-co-methacrylic acid).

15. A process as defined in claim 1, wherein the ionomer has increased gas permeability in comparison to a similarly formed ionomer that is not subjected to the energy.

16. A process as defined in claim 1, further comprising the step of oxidizing the broken polymer chains located along the damage tracks prior to hydrolyzing the ionomer precursor polymer membrane.

17. An ionomer membrane comprising:
a membrane comprising an ionomer, the ionomer containing ionic groups and non-polar polymer chains, the membrane having a thickness, the membrane defining damage tracks, the damage tracks extending through at least a portion of the thickness of the membrane, the ionic groups being clustered along the damage tracks.

18. An ionomer membrane as defined in claim 17, wherein the membrane contains damage tracks in an amount sufficient to increase the conductivity of the membrane.

19. An ionomer membrane as defined in claim 17, wherein the ionic groups comprise sulfonate groups or carboxylate groups.

20. An ionomer membrane as defined in claim 17, wherein at least a portion of the damage tracks extend through at least about 40% of the thickness of the membrane.

21. An ionomer membrane as defined in claim 17, wherein at least a portion of the damage tracks extend all the way through the thickness of the membrane.

22. An ionomer membrane as defined in claim 17, wherein the ionomer comprises a perfluorinated polymer containing sulfonate groups.

23. An ionomer membrane as defined in claim 17, wherein the ionomer comprises a copolymer of a vinyl hydrocarbon and a vinyl carboxylic acid containing carboxylate groups.

24. An ionomer membrane as defined in claim 17, wherein the membrane comprises a film.

25. An ionomer membrane as defined in claim 17, wherein the membrane comprises a coating.

26. An ionomer membrane as defined in claim 17, wherein the ionomer comprises a poly(ethylene-co-methacrylic acid).

27. An ionomer membrane as defined in claim 17, wherein the membrane comprises a contact lens.

28. A contact lens as defined in claim 27, wherein the channels formed along the damage tracks contain therapeutic agents or dyes.

29. An ionomer membrane as defined in claim 17, wherein the membrane comprises an ionic membrane.

30. An ionomer membrane as defined in claim 29, wherein the ionic membrane is incorporated into a fuel cell.

31. An ionomer membrane as defined in claim 29, wherein the ionic membrane is incorporated into an electrolysis cell.

32. An ionomer membrane as defined in claim 17, wherein the membrane comprises a coating on a wound dressing, the coating being vapor and gas permeable while being liquid impermeable.

33. An ionomer membrane as defined in claim 17, wherein the membrane contains damage tracks in an amount sufficient to increase the gas permeability of the membrane.

34. An ionomer membrane as defined in claim 17, wherein the damage tracks comprise broken polymer chains in association with peroxy free radical groups.

35. An ionomer membrane as defined in claim 17, wherein the membrane comprises a coating on an article of clothing, the coating being vapor and gas permeable while being liquid impermeable.
